(19) United States
(12) Patent Application Publication
(10) Pub. No.: US 2023/0018934 A1
(43) Pub. Date: Jan. 19, 2023

(54) METHOD OF IN VIVO ADMINISTRATION OF THE CODING SEQUENCE OF THE SIRT6 GENE VIA ADENO-ASSOCIATED VIRUS

(71) Applicant: GENFLOW BIOSCIENCES SRL, Charleroi (BE)

(72) Inventor: Eric LEIRE, Watermael-Boitsfort (BE)

(73) Assignee: GENFLOW BIOSCIENCES SRL, Charleroi (BE)

(21) Appl. No.: 17/865,688

(22) Filed: Jul. 15, 2022

Related U.S. Application Data
(60) Provisional application No. 63/222,557, filed on Jul. 16, 2021.

(51) Int. Cl. A61K 48/00; C12N 7/00; C12N 15/86; C12N 9/10

(52) U.S. Cl. CPC A61K 48/0066; C12N 7/00; C12N 15/86; C12N 9/1029; A61K 48/0025; A61K 48/0075; C12N 2750/14142; C12N 2750/14123; A61K 38/00

(57) ABSTRACT

Methods for in vivo administration of the coding sequence of the sirt6 gene. In particular, methods that include the administration of adeno-associated virus vectors or recombinant adeno-associated virus vectors including the coding sequence of the sirt6 gene.

### FIELD

The present invention relates to a use of in vivo systemic or targeted mode of administration of adeno associated virus for an episomal sustained and transient expression of SIRT6 for the purpose of extending lifespan and improving health span.

### BACKGROUND

Many attempts have been made to extend lifespan in Humans. But, so far, only activation of regulator genes has been shown to significantly increase lifespan. The activation of aging regulator genes had been done either by calorie restriction or by use of chemicals that activate regulator genes such as Sirtuin genes.

It has been demonstrated that SIRT6 gene promotes longevity in male mice, suppresses aging phenotypes and induces apoptosis in cancer cells (Kugel S. et al., Cell. 2016; 165, 1401-1415). Transgenic male mice overexpressing SIRT6 showed a significantly longer lifespan than wild-type mice by 16% (Kanfi Y. et al., Nature. 2012; 483(7388):218-21). In addition, SIRT6 transgenic mice express lower serum levels of IGF1, higher levels of IGF-binding protein 1, and altered phosphorylation levels of major components of IGF1 signaling (Diana van Heemst, Aging Dis. 2010; 1(2): 147-157). On the opposite, SIRT6-deficient mice lived shorter than controls (Kawahara TL. et al., Cell. 2009; 136:62-74). SIRT6-Knock-out mice have a reduced capacity to repair damaged DNA, and have a life expectancy 0f 21 days instead of 2 years (Mostoslaysky R. et al., Cell. 2006; 124, 315-329).

Taken together, these results support that the SIRT6 gene plays a major role in extending life span. However, no attempt has been made to deliver extra copies of SIRT6 gene in Human.

Genetic manipulations such as artificial insertion of regulator genes to the genome have been reserved to animal models and are impossible in Human. There are multiple problems with integrating methodologies of effecting protein expression. For example, introduced DNA can integrate into host cell genomic DNA at some frequency, resulting in alterations and/or damage to the host cell genomic DNA. Alternatively, the heterologous deoxyribonucleic acid (DNA) introduced into a cell can be inherited by daughter cells (whether or not the heterologous DNA has integrated into the chromosome) or by offspring.

One way to extend the lifespan of a living cell—and by extension possibly the organ, tissue or entire organism—is to repair damage in addition to preventing damage. Providing extra copies of SIRT6 gene which control the cellular repair mechanisms, may effectively extend the lifespan of a cell. This may take several forms: extending the lifespan of a cell which is damaged or injured by properly repairing that damage and/or by causing the cell to live longer or replicate itself longer than it would have occurred naturally.

The SIRT6 gene codes for a histone deacetylase that cleaves the acetyl chemical tags from histones. As a result, the DNA bundles up and is prevented from being read.

SIRT genes convert damaged euchromatin (DNA that can be read) into heterochomatin (DNA that cannot be read).

### SUMMARY

The present invention relates to methods for in vivo administration of the coding sequence of the SIRT6 gene. In particular, the present invention relates to methods comprising the administration of adeno-associated virus vectors or recombinant adeno-associated virus vectors comprising the coding sequence of the SIRT6 gene.

### DETAILED DESCRIPTION

In one embodiment, the present invention is directed to methods for extending the lifespan by providing non-integrating extra copies of the coding sequence of a regulator human gene SIRT6 (wild type or variants of the coding sequence of the gene).

The present disclosure relates to methods and compositions for extending lifespan. In one aspect of the invention, a method for extending the life of an organism is provided. The method includes administering an effective number of extra copies of regulator gene SIRT6 through adeno-associated virus vectors. The SIRT6 gene limited size (8,941 base pairs with a coding DNA sequence of thousand base pairs) allows a reliable significant episomal expression of the coding sequence of the SIRT6 by adeno-associated virus vectors (including self-complementary adeno associated virus and CpG-depleted Adeno-Associated Virus). The expression of the coding sequence of the SIRT6 gene was assessed both in vitro and in vivo. The in vitro assessment of the coding sequence of the SIRT6 gene expression was performed using deacetylation assays with SIRT6 proteins. The acetylation status of SIRT6 targets was assayed with acetyl-specific antibodies. In vitro deacetylation reactions were performed as described in (Gil et al., 2013; Michishita et al., 2008). Histone acetylation levels after reactions were detected using western blot analysis with acetylation specific antibodies. The in vitro expression of the coding sequence of the SIRT6 gene was performed using UV induced damage to human cell lines and SIRT6 knockout Human cell lines. In addition, an in vivo assessment of the coding sequence of the SIRT6 gene expression was performed in mice (CD-1 mice from Charles River Laboratories) and SIRT6 knockout mice.

In one embodiment, the present invention is directed to methods for increasing health span by providing non-integrating extra copies of the coding sequence of a regulator gene SIRT6.

The present disclosure relates to methods and compositions for delaying the onset of age-related diseases in individuals in need thereof. In one embodiment, the method applies to Human. In another embodiment, the method applies to animals (veterinary use). In one aspect of the invention, a method for extending the life of an organism is provided. The method includes administering an effective number of extra copies of the coding sequence of the regulator gene SIRT6 through adeno-associated virus vectors. The SIRT6 gene limited size SIRT6 (8,941 base pairs with a coding DNA sequence of thousand base pairs) allows a reliable significant episomal expression of SIRT6 by adeno-associated virus vectors.

In one embodiment of the present invention, the adeno-associated virus can be administered by a systemic intravenous infusion.

In a further aspect, the invention relates to the use of a intra muscular injection of the invention for extending lifespan and for delaying the onset of age-related diseases in individuals in need thereof.

In a further aspect, the invention relates to the use of intramuscular injections coupled with electroporation. In a further aspect, the invention relates to the use of an inhaled formulation for delivery to the lung via nebulization. In a further aspect, the invention relates to the use of a topical formulation for delivery to the skin fibroblasts (and other cells from the skin).

In a further aspect, the present invention relates to a composition comprising of the coding sequence of the SIRT6 gene and adeno-associated virus administered locally to an organ for delaying the onset of age-related damage to the organ.

In one embodiment of the present invention, the adeno associated virus use a CMV promotor gene. In a further aspect, the invention relates to the use of different promotor genes in order to direct expression of the SIRT6 protein to different tissues.

In one embodiment of the present invention, the coding sequence of the sirtuin 6 gene is from Human origin (wild type and several variants of coding sequence of the human sirtuin 6 gene).

In a further aspect, the invention relates to the use of the coding sequence of the sirtuin 6 gene from Strongylocentrotus franciscans. In a further aspect, the invention relates to the use of the coding sequence of the sirtuin 6 gene from Strongylocentrotus franciscans. In a further aspect, the invention relates to the use of the coding sequence of the sirtuin 6 gene from Arctica islandica. In a further aspect, the invention relates to the use of the coding sequence of the sirtuin 6 gene from Balaena mysticetus. In a further aspect, the invention relates to the use of the coding sequence of the sirtuin 6 gene from Cypinus carpio. In a further aspect, the invention relates to the use of the coding sequence of the sirtuin 6 gene from Turritopsis dohrnii Jellyfish. In a further aspect, the invention relates to the use of the coding sequence of the sirtuin 6 gene from different mammal with longer lifespan than human. In a further aspect, the invention relates to the use of synthetic sirtuin 6 gene derived from different mammal with longer lifespan than human.

In one embodiment, the present invention is directed to methods for extending the lifespan by providing non-integrating extra copies of the coding sequence of a regulator gene SIRT6.

In one embodiment, the present invention is directed to methods for extending the Health span (preventing aged-associated diseases such as neurodegenerative diseases, type 2 diabetes or cardio-vascular diseases) by providing non-integrating extra copies of the coding sequence of a regulator gene SIRT6.

The present disclosure relates to methods and compositions for extending lifespan. In one aspect of the invention, a method for extending the life of an organism is provided. The method includes administering an effective number of extra copies of regulator gene SIRT6 through adeno-associated virus vectors. The coding sequence of the SIRT6 gene limited size (thousand base pairs) allows a reliable significant episomal expression of SIRT6 by adeno-associated virus vectors.

The present disclosure also relates to a method for extending the lifespan of a subject comprising the administration of a composition comprising adeno-associated virus vectors or recombinant adeno-associated virus vectors comprising an effective number of non-integrating extra copies of the coding sequence of the sirt6 gene or a variant thereof to said subject operably linked to at least one enhancer and at least one promoter.

1. A method for extending the lifespan of a subject comprising the administration of an effective number of non-integrating extra copies of the coding sequence of the sirt6 gene or a variant thereof to said subject.

2. The method according to claim 1, wherein the extra copies of the coding sequence are comprised in adeno-associated virus vectors (AAV) or recombinant adeno-associated virus vectors (rAAV).

3. The method according to claim 2, wherein the adeno-associated virus vectors or recombinant adeno-associated virus vectors are selected from the group consisting of AAV serotype 1, serotype 2, serotype 3, serotype 4, and serotype 5.

4. The method according to claim 2, wherein the adeno-associated virus vectors or recombinant adeno-associated virus vectors are administered by systemic intravenous infusion.

5. The method according to claim 2, wherein the adeno-associated virus vectors or recombinant adeno-associated virus vectors are administered by intra muscular injection.

6. The method according to claim 2, wherein the adeno-associated virus vectors or recombinant adeno-associated virus vectors are administered by intramuscular injection coupled with electroporation.

7. The method according to claim 2, wherein the adeno-associated virus vectors or recombinant adeno-associated virus vectors are administered by inhalation.

8. The method according to claim 2, wherein the adeno-associated virus vectors or recombinant adeno-associated virus vectors are administered by topical administration.

9. The method according to claim 2, wherein the adeno-associated virus vectors or recombinant adeno-associated virus vectors are administered by local administration to an organ.

10. The method according to claim 2, wherein the non-integrating extra copies of the coding sequence of sirt6 are operably linked to at least one promotor.

11. The method according to claim 2, wherein the non-integrating extra copies of the coding sequence of sirt6 are operably linked to at least one enhancer.

12. The method according to claim 1, wherein said sirt6 gene or variant thereof is from human origin.

13. The method according to claim 1, wherein said sirt6 gene or variant thereof is from a mammal different than human.

14. The method according to claim 1, wherein said sirt6 gene or variant thereof is from Strongylocentrotus franciscans, Arctica islandica, Balaena mysticetus, Cypinus carpio or Turritopsis dohrnii Jellyfish.

15. The method according to claim 1, wherein said sirt6 gene or variant thereof is synthetic.

16. The method according to claim 1, wherein the subject is a human.

17. The method according to claim 1, wherein the subject is an animal.

18. A method for increasing health span of a subject comprising the administration of non-integrating extra copies of the coding sequence of the sirt6 gene or a variant thereof to said subject.

19. The method according to claim 18, wherein increasing health span means preventing aged-associated diseases such as neurodegenerative diseases, type 2 diabetes or cardio-vascular diseases.

20. A method for delaying the onset of age-related diseases of a subject comprising the administration of non-integrating extra copies of the coding sequence of the sirt6 gene or a variant thereof to said subject.
